(19) United States
(12) Patent Application Publication
(10) Pub. No.: US 2024/0085413 A1
(43) Pub. Date: Mar. 14, 2024

(54) METHODS, DEVICES AND COMPOSITIONS FOR THE DISCRIMINATION OF PATHOGENS BASED ON PATTERNS OF VOLATILE COMPOUNDS

(71) Applicant: Monell Chemical Senses Center, Philadelphia, PA (US)

(72) Inventors: Bruce A. Kimball, Jenkintown, PA (US); Gary K. Beauchamp, Philadelphia, PA (US); Patrick Lennon Millet, Silver Spring, MD (US)

(21) Appl. No.: 18/261,625

(22) PCT Filed: Jan. 18, 2022
(86) PCT No.: PCT/US2022/012761

§ 371 Date: Jul. 14, 2023

Related U.S. Application Data
(60) Provisional application No. 63/139,515, filed on Jan. 20, 2021.

(51) Int. Cl. G01N 33/569; G01N 33/543

(52) U.S. Cl. CPC G01N 33/56911; G01N 33/54386; G01N 33/56983

(57) ABSTRACT

Assays and diagnostic devices for rapidly distinguishing between bacterial or viral sources of infection in a biological sample from mammalian subject are provided. A biological sample from said subject is contacted with a diagnostic reagent that detects at least one, or a pattern of multiple, volatile organic compounds (VOC). Detection of the VOC or a pattern of said VOC indicates a single source of infection selected from a Gram-positive bacterial infection, a Gram-negative bacterial infection, or a viral infection. In one embodiment, a dipstick diagnostic device permits rapid discrimination between bacterial or viral infection based upon the reaction of a detectable VOC reactive compound and optional label positioned on the dipstick surface.

This invention was made with government support under grant numbers DC000014 and DC011735-01 awarded by the National Institutes of Health. The government has certain rights in this invention.

### BACKGROUND OF THE INVENTION

Alterations of the volatile metabolome (the collection of endogenous and exogenous volatile molecules present in secretions and other emanations associated with an organism) that occur in response to inflammation can be detected by conspecifics, (i.e., another member of the same species referenced) and chemometric analyses. It is well established that the bodily odor volatile profile is altered during illness. Multiple studies have demonstrated that patients with cancer (Bajtarevic et al. 2009; Bernabei et al. 2008; Jezierski et al. 2015), intestinal infection (Burdette and Bernstein 2007), diabetes (Greiter et al. 2010), tuberculosis (Mahoney et al. 2013; Mgode et al. 2012), and other conditions (Bijland et al. 2013) exhibit alteration of body odor. As such, there has been significant interest in recent years in developing animal biosensors (animal trained to identify an odor) to identify illnesses. Such investigations rarely reveal the presence of novel volatile metabolites in patient samples (Amann et al. 2014; Kimball 2016), with the notable exception of exhaled breath during pulmonary infection (Phillips et al. 2012; van Oort et al. 2018). Instead, illness regularly results in alteration of the pattern of volatile metabolites present in the healthy state. The origin of these alterations is currently unknown. Furthermore, the specificity of these volatile patterns with respect to individual illnesses has not yet been established. In fact, little is known about the mechanism(s) responsible for alterations of the volatile metabolome.

Differential diagnoses can be challenging in certain medical conditions. For example, in sepsis and systemic inflammatory response syndrome (SIRS). Delaying antimicrobial treatment for septic patients by a matter of hours significantly increases the risk of mortality (Barie et al. 2005; Kumar et al. 2006; Morrell et al. 2005). As a result, standard emergency medical intervention for patients suspected of sepsis is immediate treatment with broad spectrum antimicrobials, prior to any microbiological testing confirming the identity or existence of the responsible pathogens (Angus and van der Poll 2013; Dellinger et al. 2013). Lack of differential diagnosis in these conditions, and others, leads to unnecessary treatments with antibiotics and antimicrobials not targeted to the specific causative pathogen. This unnecessary treatment can subject patients to potentially toxic side effects and can contribute to the rise of treatment-resistant microorganisms.

The differential diagnosis of febrile diseases (i.e., diseases characterized by fever) is specially challenging in low-resource environments. In particular, it is often difficult to determine whether there is a bacterial or a non-bacterial cause for a child who presents with a fever. For example, following exclusion of malaria as the cause of fever in areas where malaria is endemic, identifying whether or not febrile illness with fever is caused by a bacterium or virus is a key first step for guiding treatment. Routine default treatment with antibiotics is particularly common in low and middle-income countries. In addition to the higher fatality rates from drug-resistant infections, children with drug resistant infections require more expensive treatments which can have more adverse effects and excessive and prolonged hospitalizations.

Diagnostic tests for febrile diseases have been broadly classified into pathogen-specific and pathogen non-specific tests (Bhaskaran et al.). Pathogen-specific assays have the goal of diagnosing the specific disease that is causing the presenting fever that may be accompanied by other symptoms such as cough, diarrhea, and general malaise. In contrast, pathogen non-specific assays are markers that only differentiate bacterial from non-bacterial causes of fever. The vast majority of biomarkers currently being evaluated to discriminate bacterial from other causes of acute febrile illness involve hematological factors, inflammatory molecules, cytokines, cell surface and metabolic markers, and various combinations of these markers (Dittrich et al.; Kapasi et al.). These biomarkers are typically collected from blood and plasma. While molecular techniques for diagnosis of both bacteria and viruses hold great promise for specific disease diagnosis, they too often suffer from high technical barriers in terms of cost as well as complexity and need for advanced technical support and know-how.

A continuing need in the art exists for new and effective tools and methods to detect the causes of disease and enable quick and accurate determination of suitable treatments.

### SUMMARY OF THE INVENTION

The inventors have determined new and effective tools and methods for rapid diagnosis of, and discrimination between the broad classes of bacterial or viral infection based upon the rapid detection of certain volatile organic compounds (VOCs) and exploiting patterns of VOCs associated with such broad classes of infections.

In one aspect, an assay is provided for rapidly distinguishing a source of infection in a mammalian subject. This assay involves contacting a biological sample from said subject with a diagnostic reagent that detects at least one, or a pattern of multiple, volatile organic compounds (VOC). Detection of one or more of such VOCs, or a pattern or profile of said VOCs indicates a source of infection. In one embodiment, the method can distinguish between a bacterial infection or a viral infection. In another embodiment, the method can distinguish between a Gram-positive or Gram-negative bacteria, without identifying the specific bacterium. In one embodiment, the detection is of a single broad class of infection. In one embodiment, the infection is one that causes a febrile disease.

In another aspect, an assay is provided for rapidly distinguishing a source of infection in a mammalian subject by detecting in a biological sample and comparing the levels of one, two or all three of the VOCs 2-ethyl-1-hexanol, acetophenone, and 3-methyl-4-hexen-2-one. In one embodiment, the levels of these VOCs are distinguishable by comparison to known levels in a standard for a bacterial infection and a standard for viral infection.

In still another aspect, a diagnostic device configured for detection of a Gram-positive bacterium, a Gram-negative bacterium or a virus in a biological test sample. In one embodiment, the test sample is urine. In other embodiments, other liquid samples may be tested. In one embodiment, the device is configured for detection of a selected VOC or pattern or profile of VOC in a test sample via a dipstick assay.

Still other aspects and advantages of these compositions and methods are described further in the following detailed description of the preferred embodiments thereof.

### BRIEF DESCRIPTION OF THE DRAWINGS

FIG. 1 is a simplified schematic of MyD88-mediated and TRIF-mediated inflammatory signaling pathways. Shared pathway responses are marked with arrows.

FIG. 2A is a graph showing relative change in bodyweight for each mouse, normalized to its own day 0. Mice receiving phosphate buffered saline (PBS) are indicated by diamonds. Mice receiving 2 mg/kg Lipopolysaccharide (LPS) are indicated by squares. Mice administered 0.01 mg/Kg LPS are indicated by circles. Bars indicate standard error.

FIG. 2B is a bar graph showing tumor necrosis factor (TNF) found in serum for the same groups of mice as in FIG. 2A, as determined by ELISA. In each of the two sets, the leftmost bar is PBS-treated mice, the middle bar is LPS 2 mg/kg-treated mice; and the rightmost bar is the lower dosage of LPS-treated mice. *: p<0.05 compared to LPS at same time point; **: p<0.01; Determined by student's t-test.

FIG. 3A is a graph showing relative change in bodyweight for each treated mouse, normalized to its own day 0. Bars are standard error. Diamonds represent mice administered PBS. Squares represented mice administered a synthetic analog of dsRNA known as polyinosinic-polycytidylic acid (Poly(I:C)). dsRNA is associated with viral infections. Triangles represent mice administered a lipopeptide from Gram-positive bacteria (PAM3). Circles represent mice administered LPS.

FIG. 3B is a bar graph showing TNF found in serum, as determined by ELISA. **: p<0.01 compared to LPS at same time point; ***: p<0.001; Determined by student's t-test. The order of bars, left to right, are PBS-treated mice, Poly(I:C) treated mice, Pam3 treated mice and LPS treated mice as in FIG. 3A.

FIG. 3C is a bar graph showing IL-1B found in serum, as determined by ELISA. **: p<0.01 compared to LPS at same time point; ***: p<0.001; Determined by student's t-test. The order of bars, left to right, in each time period are PBS-treated mice, Poly(I:C) treated mice, Pam3 treated mice and LPS treated mice as in FIG. 3A.

FIG. 4 is a graph showing mean responses of biosensors in Y-maze apparatus. Bars represent 95% confidence intervals. Black bar is rewarded training trials. Shaded bar is unrewarded generalization to novel donor (i.e., a mouse receiving treatment from which urine is collected) treated with LPS or PBS. White bar is unrewarded generalization to 0.01 mg/kg LPS-treated mouse urine odor. All responses were significantly different than chance (50%, p<0.001).

FIG. 5 is a graph showing mean responses of biosensors in Y-maze apparatus. Bars represent 95% confidence intervals. Black bar is rewarded training trials. Shaded bar is unrewarded generalization to novel donor treated with LPS or PBS. White bars are unrewarded generalizations to Pam3CSK4 or Poly(I:C)-treated mouse urine odor. ***: p<0.001 that distribution differs from 50%, as determined by 2-tailed binomial hypothesis testing.

FIG. 6 is a radar plot of the relative expression of the seven predictors used in LDA modeling. Expression is centered and scaled.

FIG. 7 is a schematic showing that pathogens are recognized by toll-like receptors to initiate a cascade of chemokines, cytokines, and other metabolites. Resulting volatile metabolites may be used to determine the source of the infection. Lipopolysaccharide (LPS), Pam3CSK4, and Poly(I:C) were used to cause alterations of urinary volatiles in mice. Both trained biosensors and chemometric models determined these volatile patterns to differ (see arrow labeled A). Trained biosensors found these volatile patterns to differ, while chemometric models found them to be similar (see arrow labeled B).

FIG. 8 is a radar plot of the relative expression of three VOCs indicating the pattern of detection of these VOCs in Gram positive bacterial infection, Gram negative bacterial infection, and viral infection. The Poly(I:C) (green line) represents viral infection. The LPS (blue line) represents Gram negative infection. The Pam3CSK4 (red line) represents Gram positive infection.

### DETAILED DESCRIPTION

The inventors provide assay methods and devices that enable rapid differential medical diagnosis of febrile diseases by analysis of the volatile metabolome. In particular, the data provided herein support that bacterial infections can be differentiated from viral infections by use of these assays and devices, resulting in the ability to drastically improve antibiotic drug treatments. To the inventors' knowledge, no one has previously demonstrated that broad classes of infections with gram-negative bacteria, gram-positive bacteria, or viruses can be discriminated from each other on the basis of volatile odors.

Using a model system where mouse urinary metabolites are altered by treatment with lipopolysaccharide (LPS), we determined that alteration of body odor volatiles vary according to the type of pathogen responsible for inducing the inflammation. Such patterns do not appear to vary with the intensity of inflammation. The data below demonstrate that different immunogens engage different immune signaling pathways and alterations of body odor volatiles resulting from inflammation contain information about the broad type of pathogen that instigated the inflammation. This information was exploited to develop useful rapid assays and diagnostic devices.

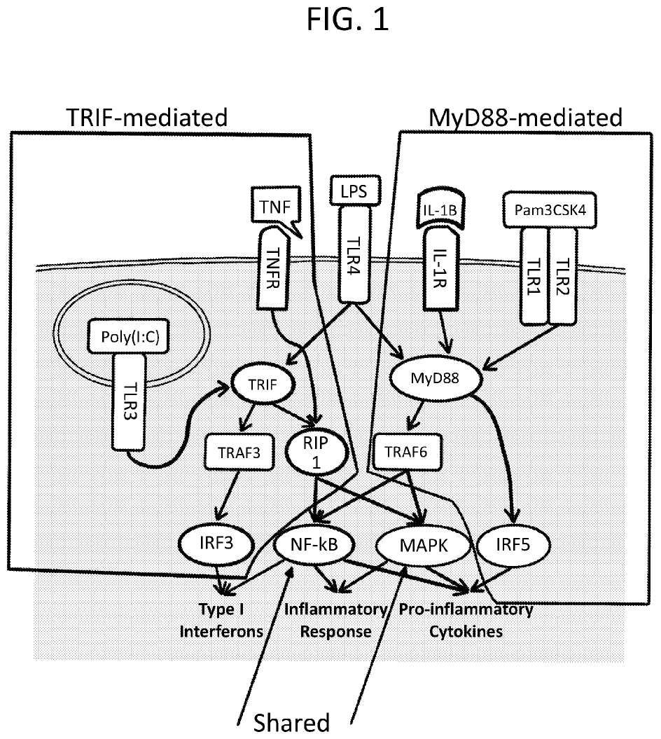

We hypothesized that distinct body odor volatile profiles result from differential Toll-like Receptor (TLR) recognition and/or stimulation of a Toll/IL-1 receptor (TIR) domain-containing protein that associates with TLRs via TIR:TIR domain interactions and facilitates downstream signaling (MyD88) and Toll/IL-1R domain-containing adaptor-inducing IFN-β (TRIF) pathways. As described in the examples below, we treated murine urine donors with several molecules known to activate cellular immunity via interaction with specific TLRs (see FIG. 1). Collectively, these molecules (which are not pathogenic themselves) are known as pathogen-associated molecular patterns (PAMPs). In addition to using LPS, which activates both MyD88 and TRIF via recognition by TLR4 (responsible for recognition of gram-negative bacteria), we employed two additional PAMPs. One, the synthetic lipopeptide Pam3CSK4 (Aliprantis et al. 1999; Brightbill et al. 1999) activates MyD88 by way of the TLR2/TLR1 heterodimer (responsible for recognition of gram-positive bacteria). The other, synthetic double-stranded RNA poly(I:C), is a ligand for TLR3 (responsible for recognition of viral RNA) and activates TRIF (Alexopoulou et al. 2001). We used trained animal biosensors and headspace gas chromatography/mass spectrometry (GC/MS) toward understanding the mechanism(s) for alteration of the volatile metabolome to ascertain that the volatile metabolome may serve as a tool for diagnosing febrile diseases. The results of these investigations provide the basis for the assays and devices described herein.

### I. Components and Definitions

Technical and scientific terms used herein have the same meaning as commonly understood by one of ordinary skill in the art to which this invention belongs and by reference to published texts, which provide one skilled in the art with a general guide to many of the terms used in the present application. The definitions contained in this specification are provided for clarity in describing the components and compositions herein and are not intended to limit the claimed invention.

As used herein, the terms “Volatile Compounds” or “Volatile Organic Compounds” refers to certain carbon-based compounds, with high vapor pressure and low boiling points. VOCs are produced within the body as a result of metabolic processes from the host, from microbial pathogens, or resulting from a host response to pathological processes, such as infection or inflammation. VOCs in certain samples such as urine may also be intermediate or end products of metabolic pathways. See, e.g., Dospinescu et al, 2020. Of a total of about 1800 identified volatile organic compounds identified in mammals, about 872 are identified in breath, about 359 are identified in saliva, about 154 are identified in blood, about 279 are identified in urine and about 381 are identified in feces. Among the VOCs useful in the present assay and as targets for the diagnostic devices here include acetophenone (C₆H₈O), also known as 1-phenylethanone, acetylbenzene, benzoyl methide, methyl phenyl ketone or phenyl methyl ketone. In another embodiment, as suitable VOC is 3-methyl-4-hexen-2-one (C₇H₁₂O), also known as 3-methylhex-4-en-2-one, 4-Hexen-2-one,3-methyl-, 3-Methyl-4-Hexen-2-one. In still another embodiment, a suitable VOC for use in the assays and devices herein is 2-ethyl-1-hexanol (C₈H₁₈O), also known as 2-ethylhexyl alcohol. For still other suitable VOC targets or biomarkers, see e.g., Amann A, et al. 2014 and Table 2 of Dospinescu et al 2020, among other publications incorporated herein by reference.

The phrase “Volatile Organic Compound binding molecule” or “VOC binding molecule” refers to a compound or molecules that can detect the presence or absence or level of different VOCs, a VOC biomarker receptor, a related VOC or portion thereof in a test sample or on a test surface. Generally, the VOC binding molecules disclosed herein can bind with or identify a gram-positive, gram-negative, or viral infection. In certain embodiments, VOC binding molecules can coat a testing apparatus for detection of a broad class of pathogen or microbe. The term “Chemical reactants of VOCs or substances” as used herein refers to a molecule that binds or complexes, or reacts chemically with a VOC, a VOC biomarker receptor, a related VOC or portion thereof. Such molecules, depending upon the assay method employed for detection and measurement of the level of VOC, can include other chemical compounds, molecular forms or peptides, such as an antibody, antibody mimic or equivalent that binds to or complexes with a VOC biomarker, or a fragment or portion thereof. In certain embodiments, in which the VOC biomarker is to be evaluated, the ligand can be a nucleotide sequence, e.g., polynucleotide or oligonucleotide, primer or probe to its receptor on cells in the biological sample. VOC-reactive compound or substance is a chemical reactant or an enzyme, or a chemical reactant or enzyme bound to a signal molecule. In one embodiment, a VOC-reactive compound or substance is 2,4-dinitrophenylhydrazine. In another embodiment, a VOC-reactive compound or substance is ceric nitrate. In another embodiment, a VOC-reactive compound or substance is Schiff's reagent. In another embodiment, a VOC-reactive compound or substance is Fluorescamine. In another embodiment, a VOC-reactive compound or substance is Ehrlich's reagent. Still other reactants with the selected VOC biomarkers may be selected from suitable chemical reactants, based on the identity of the VOC.

“Patient” or “subject” as used herein means a mammalian animal, including a human, a veterinary or farm animal, a domestic animal or pet, and animals normally used for clinical research. In one embodiment, the subject of these methods and compositions is a human.

“Biological sample” as used herein means any biological fluid or tissue that contains the VOC biomarkers described herein. In certain embodiments the test samples include the bodily fluids and tissues of a subject. In certain embodiments the bodily fluids and tissues are urine, blood, saliva, feces, or air exhaled by a subject. The most suitable samples for use in the methods and with the compositions are samples which require minimal invasion for testing, e.g., urine, saliva, tears, and blood samples, including serum, plasma, and whole blood. It is also anticipated that other biological fluids, such as nasal, vaginal or cervical secretions, and ascites fluids or peritoneal fluid may be similarly evaluated by the methods described herein. Also, circulating tumor cells or fluids containing them are also suitable samples for evaluation in certain embodiments of this invention. Such samples may further be diluted with saline, buffer or a physiologically acceptable diluent. Alternatively, such samples are concentrated by conventional means.

As used herein, the phrase “coating” or “coated” is generally meant a layer of molecules or material formed on an outermost or exposed layer of a surface.

Lipopolysaccharide (LPS) is an integral component of gram-negative bacterial cell walls. In rodents, inflammation induced by LPS or inflammatory cytokines impacts the body odor volatile profile (Arakawa et al. 2011a; Arakawa et al. 2010; Arakawa et al. 2011b; Gervasi et al. 2018; Kimball et al. 2014a; Millet et al. 2018). This change in body odor may cause healthy conspecifics to avoid the odor of the sick individual (Arakawa et al. 2009a; Arakawa et al. 2009b). Similar conspecific avoidance behavior was observed in rodents that were parasitized or infected with respiratory virus (Kavaliers and Colwell 1995; Kavaliers et al. 1998; Penn et al. 1998). LPS is frequently used in models of inflammation, owing to its ability to produce a robust innate immune response (Baldwin 1996; Beeson 1947; Geppert et al. 1994; Lu et al. 2008; Raetz and Whitfield 2002). It is one of several pathogen-associated molecules that are recognized by toll-like receptors (TLRs). LPS is recognized by TLR4.

Toll-like Receptors (TLRs) recognize different molecular patterns that are characteristic to other pathogens, like triacylated lipopeptides (found in many bacteria), flagellin (found in bacterial flagellum), double stranded RNA (found in RNA viruses) or unmethylated CpG DNA (found in bacterial and viral genomes) (Lu et al. 2008). While TLRs lack the versatility of the adaptive immune response, they can quickly respond to an array of common pathogenic molecules. Upon recognizing their ligand, TLRs rapidly promote an innate inflammatory response. The exact nature of this response differs somewhat depending on which TLR is activated. Most TLRs activate inflammation by way of one of two intracellular mediators: MyD88 or TRIF (FIG. 1).

Gram-Positive bacteria refer to bacteria that retain the color of the crystal violet stain in the Gram stain. This is characteristic of bacteria that have a cell wall composed of a thick layer of peptidoglycan. The Gram-positive bacteria include staphylococci (“staph”), streptococci (“strep”), pneumococci, and the bacterium responsible for diphtheria (Cornyebacterium diphtheriae) and anthrax (Bacillus anthracis).

Gram-Negative bacteria refer to bacteria that lose the crystal violet stain (and take the color of the red counterstain) in Gram's method of staining. This is characteristic of bacteria that have a cell wall composed of a thin layer of peptidoglycan. Gram-negative bacteria include most of the bacteria normally found in the gastrointestinal tract that can be responsible for disease as well as gonococci (venereal disease) and meningococci (bacterial meningitis). The organisms responsible for cholera and bubonic plague are Gram-negative.

“Reference standard” as used herein refers to the source of the reference VOC biomarker levels. The “reference standard” is preferably provided by using the same assay technique as is used for measurement of the subject's VOC biomarker levels in the reference subject or population, to avoid any error in standardization. The reference standard is, alternatively, a numerical value, a predetermined cutpoint, a mean, an average, a numerical mean or range of numerical means, a numerical pattern, a ratio, a graphical pattern or a pattern of two or more VOC derived from the same biomarker or biomarkers in a reference subject or reference population. In an embodiment, the reference standard can be an expression level of one or more VOC biomarkers or an expression profile.

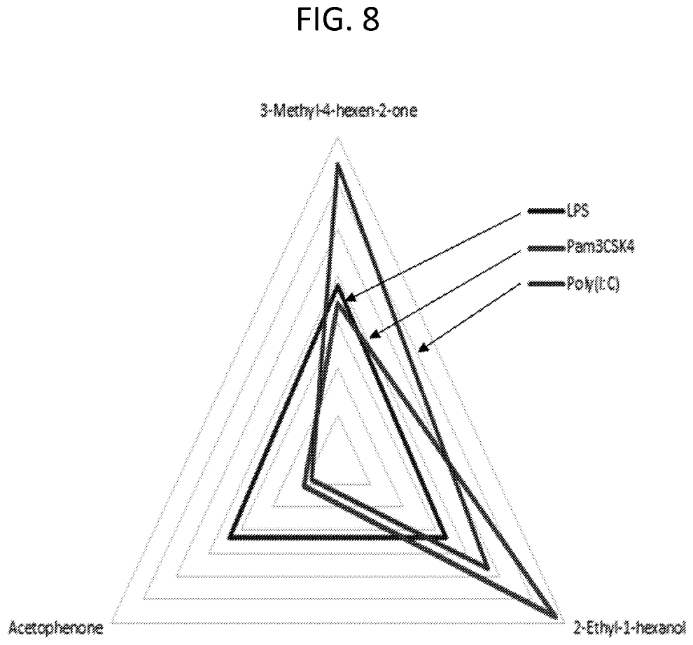

“Reference subject” or “Reference Population” defines the source of the reference standard. In one embodiment, the reference is a mammalian subject or a population of subjects having a Gram-negative infection. In yet another embodiment, the reference is a mammalian subject or population of subjects with a Gram-positive bacterial infection. In still another embodiment, the reference is a mammalian subject or a population of subjects having a viral infection. In still another embodiment, the reference is a mammalian subject or a population of subjects who has a febrile disease. In still another embodiment, the reference is a mammalian subject who has a non-infection source of fever (such as trauma). In yet another embodiment, the reference subject is the average of multiple subjects of the populations described herein, such as shown in FIG. 8.

As used herein, “labels” or “reporter molecules” are chemical or biochemical moieties or molecules that generate a detectable signal due to a reaction with the VOC biomarker or one of its metabolites or due to its association with a VOC-reactant or binding substance. “Labels” and “reporter molecules” or signals include fluorescent agents, chemiluminescent agents, chromogenic agents, quenching agents, radionucleotides, enzymes, substrates, cofactors, inhibitors, radioactive isotopes, magnetic particles, and other moieties known in the art. “Labels” or “reporter molecules” are capable of generating a measurable signal and may be covalently or noncovalently joined to a VOC-reactant compound or substance. In one embodiment, the signal/labeling component is a dye; a reactive dye; a fiber reactive dye; a monohalogentriazine dye; a dihalogentriazine dye; a 2,4,5 trihalogenopyriminidine dye; a 2,3 dihaloquinoxaline dye; a N-hydroxysulfosuccinimidyl a (sulfo-NHS) ester functionalized dye; a N-hydroxysuccinimidyl (NHS) functionalized dye; a vinyl sulfone dye; a sulfonylchloride dye; a tetrafluorophenyl ester functionalized dye; an isothiocyanate functionalized dye; and an iodoacetyl functionalized dyes. Still other signal components are chemically responsive dyes that change color when VOCs contact the dye molecules and cause a chemical reaction. See U.S. Pat. No. 8,927,104 incorporated herein by reference. In another embodiment, the label is a fluorescent probe and a quencher dye molecule, or a label selected from the group consisting of spin labels, antigen tags, epitope tags, haptens, enzyme labels, prosthetic groups, fluorescent materials, pH-sensitive materials, chemiluminescent materials, colorimetric components, bioluminescent materials, and radioactive materials. See, e.g., U.S. Pat. No. 8,609,358. Still other suitable labels or signaling compositions are known in the art. See e.g., U.S. Pat. No. 10,788,498.

By the term “solid support” or “solid substrate” is meant an array, a micro well plate, a microfluidic device, a bead, an absorptive film, a dip stick, a chemical sensor, and/or an electrical sensor or chip which permits the VOC-reactant compound or binding substance to be immobilized.

The terms “a” or “an” refers to one or more. For example, “an expression cassette” is understood to represent one or more such cassettes. As such, the terms “a” (or “an”), “one or more,” and “at least one” are used interchangeably herein.

As used herein, the term “about” means a variability of plus or minus 10% from the reference given, unless otherwise specified.

The words “comprise”, “comprises”, and “comprising” are to be interpreted inclusively rather than exclusively, i.e., to include other unspecified components or process steps. The words “consist”, “consisting”, and its variants, are to be interpreted exclusively, rather than inclusively, i.e., to exclude components or steps not specifically recited.

### II. Assays

One aspect of the methods provided herein is an assay for distinguishing a source of infection in a mammalian subject comprising contacting a biological sample from said subject with a diagnostic reagent that detects at least one, or a pattern of multiple, volatile organic compounds (VOC), wherein detection of said compound or a pattern of said VOC indicates a single source of infection selected from a Gram-positive bacterial infection, a Gram-negative bacterial infection, or a viral infection.

In one embodiment, as discussed above, the VOC is 3-methyl-4-hexen-2-one. In one embodiment, the VOC is acetophenone. In another embodiment, the VOC is 2-ethyl-1-hexanol. In still another embodiment, a pattern of VOCs is provided by the detection of levels of 3-methyl-4-hexen-2-one and acetophenone. In still another embodiment, a pattern of VOCs is provided by the detection of levels of are 3-methyl-4-hexen-2-one and 2-ethyl-1-hexanol. In still another embodiment, a pattern of VOCs is provided by the detection of levels of 2-ethyl-1-hexanol and acetophenone. In yet another embodiment, a pattern of VOCs is provided by the detection of levels of 2-ethyl-1-hexanol, acetophenone, and 3-methyl-4-hexen-2-one.

The assays may utilize steps to detect increased or decreased acetophenone levels relative to a suitable standard as stated above. In one embodiment, for classification of the sample as a Gram-negative bacterial infection, the assay permits detection of increased acetophenone relative to a reference standard for viral infection and/or a reference standard for Gram-positive infection, and/or a reference standard for a febrile disease caused by a non-infectious circumstance, e.g., trauma. An increased acetophenone level is at least 1, 2, 3, 4, 5, or 10 times more than the reference standard. A decreased acetophenone level is at least 1, 2, 3, 4, 5, or 10 times less than the reference standard.

In yet another embodiment, the assays may utilize steps to detect increased or decreased 2-ethyl-1-hexanol levels relative to a suitable standard as stated above. In one embodiment, for classification of the sample as a Gram-positive bacterial infection, the assay permits detection of increased 2-ethyl-1-hexanol relative to a reference standard for viral infection and/or a reference standard for Gram-negative infection and/or a reference standard for a febrile disease caused by a non-infectious circumstance, e.g., trauma. An increased 2-ethyl-1-hexanol level is at least 1, 2, 3, 4, 5, or 10 times more than the reference standard. A decreased 2-ethyl-1-hexanol level is at least 1, 2, 3, 4, 5, or 10 times less than the reference standard.

In yet another embodiment, the assays may utilize steps to detect increased or decreased 3-methyl-4-hexen-2-one levels relative to a suitable standard as stated above. In one embodiment, for classification of the sample as a viral infection, the assay permits detection of increased 3-methyl-4-hexen-2-one relative to a reference standard for Gram-positive bacterial infection and/or a reference standard for Gram-negative infection and/or a reference standard for a febrile disease caused by a non-infectious circumstance, e.g., trauma. An increased 3-methyl-4-hexen-2-one level is at least 1, 2, 3, 4, 5, or 10 times more than the reference standard. A decreased 2-ethyl-1-hexanol level is at least 1, 2, 3, 4, 5, or 10 times less than the reference standard.

These relationships between particular infections and their reference standard can also be used to establish a pattern for the infection of the reference standard. For example, in one embodiment, for classification of the sample as a Gram-positive bacterial infection or viral infection, the assay permits detection of decreased acetophenone relative to a reference standard.

In still another embodiment, the assay permits detection of a pattern of VOC levels is as demonstrated in FIG. 8 to discriminate between bacterial (Gram negative or Gram positive) and viral infection.

In any of the assays described herein, the biological sample can be a non-invasive biological fluid suitable for use of a dipstick assay, such as urine, saliva, tears, nasal secretions. Blood or other more invasively obtained biological samples may also be subject to an assay as described herein.

The assays may employ a diagnostic reagent comprises a compound or substance that reacts with one or more selected VOC upon contact with the sample containing the VOCs to produce a characteristic signal. In one embodiment, the signal is a color change or colorimetric signal, a fluorescent signal, an enzymatic signal or a chromogen or any one of the signals or labels described above and suitable to the diagnostic reagent or method. For example, in one embodiment, the reagent comprises a dipstick impregnated with said VOC-reactive compound or substance. As discussed above, the VOC-reactive compound or substance associated with a dipstick or other solid subtract or surface is a chemical reactant or an enzyme, or a chemical reactant or enzyme bound to a signally molecule. Although the VOC-reactive compound or substance may be selected from among known compounds or substance that reacts or binds the target VOC, in one embodiment, the VOC-reactive compound is 2,4-dinitrophenylhydrazine. In still other embodiments of the assays, the VOC-reactive compound may be one selected for the specific VOCs desired for detection and comparison as discussed herein.

As discussed in Dospinescu, V-M et al, incorporated herein by reference, there are various competing assay technologies available for the analysis of VOCs in a biological sample. The most commonly used technique is gas chromatography, coupled with mass spectrometry (GC-MS). Other methods include proton transfer reaction mass spectrometry (PTR-MS), ion mobility spectrometry (IMS), selected ion flow tube mass spectrometry (SIFT-MS), field asymmetric ion mobility spectrometry (FAIMS), gas chromatography flame ionization detection (GC-FID), and electronic noses (eNoses), which are made up of arrays of sensors, e.g., metal oxide (MOS), piezoelectric, or conducting polymer (CP). However, a dipstick assay is preferred for simplicity.

### III. Diagnostic Devices

In another aspect, a diagnostic device is configured for detection of a Gram-positive bacterium, a Gram-negative bacterium or a virus in a test sample as described herein. In one embodiment, the device is configured for detection of a selected VOC or pattern of VOC in a test sample via a competition assay, a sandwich assay, a displacement assay, an electrochemical detection, a colorimetric detection, an enzymatic detection, an enzyme-linked immunosorbent assay (ELISA), or a dipstick assay. The device, in one embodiment, comprises a compound or substance that reacts with one or more selected VOC upon contact with the sample containing the VOCs to produce a characteristic signal. The device may have associated on a surface thereof a VOC-reactive compound or substance. The compound or substance may be conjugated, attached or immobilized onto a solid substrate. In certain embodiments, at least a portion of the VOC reactive compound or substance is conjugated or associated with a detectable label or signal component.

In one embodiment, such a device includes a sample pad; the VOC reactive compound or substance immobilized on a first surface; and wherein the sample pad is in operable fluid communication with the first surface. In certain embodiments, the first surface is a porous surface. The first surface may comprise a nitrocellulosic material, polyvinylidene fluoride (PVDF), polyethylene material, nylon, cellulose acetate, polyester material, polyethersulfone (PES), or polysulfone. In still another embodiment, the surface is positioned in an enclosed housing. See, e.g., U.S. Pat. Nos. 10,768,185 or 10,768,171 for further description of such a device.

In some embodiments, the diagnostic device or testing apparatus is a dipstick and/or a test strip. For example, a dipstick and/or a test strip can include at least one test area coated with one or more VOC binding molecules. The dipstick and/or a test strip can be in any shape and/or in any format, e.g., a planar shape such as a rectangular strip or a circular disk, or a curved surface such as a stick. Alternatively, a continuous roll can be utilized, rather than discrete test strips, on which the test area(s) and optionally reference area(s) are present in the form of continuous lines or a series of spots. In some embodiments, the dipsticks or test strips described herein can be used as point-of-care diagnostic tools for microbe or pathogen identification.

In some embodiments, the dipstick or test strip can be made of any material, including, without limitations, paper, nitrocellulose, glass, plastic, polymer, membrane material, nylon, and any combinations thereof. In one embodiment, the dipstick or a test strip can include paper. In one embodiment, the dipstick or a test strip can include nylon.

In some embodiments, the dipstick or a test strip can further comprise at least one reference area or control area for comparison with a readout signal determined from the test area. The reference area generally excludes the VOC binding molecules, e.g., to account for any background signal. In some embodiments, the reference area can include one or more known amounts of the detectable label that the VOC binding molecules in the test area encompass. In such embodiments, the reference area can be used for calibration such that the number of microbes or pathogens in a test sample can be estimated or quantified.

In still other embodiments, the dipstick/test strip can comprise multiple test areas. Each test area is coated with a VOC binding molecule that identifies a different VOC. In some embodiments, the test area is coated with a VOC binding molecule that identifies one of acetophenone, 2-ethyl-1 hexanol, and 3-methyl-4-hexen-2-one. In some embodiments, the dipstick/test strip contains two or three test areas, where each area is coated with a VOC binding molecule that binds one of acetophenone, 2-ethyl-1 hexanol, and 3-methyl-4-hexen-2-one. In some embodiments, the dipstick/test strip contains at least one additional test area that identifies an additional VOC per additional test area.

Various other methods known by those skilled in the art can be used to identify volatile organic compounds. In certain embodiments, the VOC can be identified using Gas chromatography (WO Application No. PCT/JP02/08323), mass spectrometry (U.S. patent application Ser. No. 16/077,063), (CN Patent Application No. 107462627A), Gas chromatography-mass spectrometry (KR Patent Application No. 2017/013888), (Luna, G.; Aparicio, R.; Garcia-Gonzalez, D. L. Food Chem. 2006, 97, 621-630 DOI: 10.1016/j.foodchem.2005.05.039), Chemical sensor systems (US 2020/0386730), Electronic Nose Sensors (Son, M., et al, Analytical Chemistry 2016 88 (23), 11283-11287, DOI: 10.1021/acs.analchem.6b03284), ultraviolet illumination systems (U.S. Pat. No. 10,627,378), and high performance liquid chromatography. Other known methods used to identify VOCs can be found in U.S. Pat. Nos. 9,588,084, and 5,384,262. The entire disclosure of each of these, and each disclosure cited above, is incorporated herein by reference as though set forth in full.

In one embodiment, VOC selective detectors or “artificial noses” have developed to detect and characterize gaseous samples. A multitude of technologies have implemented artificial nose functions including, but not limited to colorimetric sensor arrays, polymer arrays, mass sensitive piezoelectric substrates, surface acoustic wave (SAW) transducers, quartz crystal microbalances, functionalized carbon nanotubes and gold nanoparticles.

Vapor-selective detectors or “artificial noses” are typically based upon the production of an interpretable signal or display upon exposure to a vapor emitting substance or odorant (hereinafter sometimes referred to as an “analyte”). More specifically, typical artificial noses are based upon selective chemical binding or other molecular interactions in the interface between a detecting compound of the artificial nose and an analyte or odorant, and then transforming that chemical binding into a signal or display, i.e., signal transduction.

Polymer arrays having a single dye have been used for artificial noses. That is, a series of chemically diverse polymers or polymer blends are chosen so that their composite response distinguishes a given odorant or analyte from others. Examples of polymer array vapor detectors, including conductive polymer and conductive polymer/carbon black composites, are discussed in: M. S. Freund, N. S. Lewis, Proc. Natl. Acad. Sci. USA 92, 2652-2656 (1995); B. J. Doleman, et al, Anal. Chem. 70, 2560-2564 (1998); T. A Dickinson, J et al., Nature 382, 697-700 (1996) (polymer array with optical detection); A E. Hoyt, et al, J. Am. Chem. Soc. 117, 8672 (1995); and J. W. Grate, M. H. Abraham, Sensors and Actuators B 3, 85-111 (1991).

Other interface materials include functionalized self-assembled monolayers (SAM), metal oxides, and dendrimers. Signal transduction is commonly achieved with mass sensitive piezoelectric substrates, surface acoustic wave (SAW) transducers, or conductive materials. Optical transducers (based on absorbance or luminescence) have also been examined. Examples of metal oxide, SAM, and dendrimer-based detectors are discussed in J. W. Gardner, et al, Sensors and Actuators B 4, 117-121(1991); J. W. Gardner, et al, Sensors and Actuators B 6, 71-75 (1992); and R. M. Crooks, A. J. Ricco, Acc. Chem. Res. 31, 219-227 (1998). These devices also use a single dye.

Techniques have also been developed using a metalloporphyrin for optical detection of a specific, single gas such as oxygen or ammonia, and for vapor detection by chemically interactive layers on quartz crystal microbalances. See A. E. Baron, et al., Rev. Sci. Instrum. 64, 3394-3402 (1993); J. Kavandi, et al, Rev. Sci. Instrum. 61, 3340-3347 (1990); W. Lee, et al., J. Mater. Chem. 3, 1031-1035 (1993); A. A. Vaughan, et al, Anal Comm. 33, 393-396 (1996); J. A J. Brunink, et al, Anal. Chim. Acta 325, 53-64 (1996); C. DiNatale, et al, Sensors and Actuators B 44, 521-526 (1997); and C. DiNatale, et al., Mat. Sci. Eng. C 5, 209-215 (1998).

Other techniques include functionalized carbon nanotubes sometimes integrated into a transistor, see DNA-Decorated Carbon Nanotubes for Chemical Sensing. Cristian Staii and Alan T. Johnson, Jr, Nano Letters 2005 and functionalized gold nanoparticles see Broza, Y. Y., & Haick, H. (2013). Nanomaterial-based sensors for detection of disease by volatile organic compounds. Nanomedicine, 8(5), 785-806; Barash, O., et al (2009). Sniffing the Unique “Odor Print” of Non-Small-Cell Lung Cancer with Gold Nanoparticles. Small, 5(22), 2618-2624. See also, Sun et al and Murugathas, T et al, 2019, incorporated by reference herein.

In yet another embodiment, the diagnostic reagent for targeting the selected VOC in a biological sample includes an artificial nose based on colorimetric sensor arrays that are capable of detecting VOCs at low concentrations with a high degree of accuracy. Colorimetric sensor arrays that are capable of detecting VOCs typically contain chemically responsive dyes that change color when VOCs contact the dye molecules and cause a chemical reaction. Sensor arrays typically contain a variety of types of reactive molecules that respond to different VOCs. Examples of sensor arrays are described in, for example, U.S. Pat. No. 6,368,558, issued on Apr. 9, 2002, titled Colorimetric Artificial Nose Having an Array of Dyes and Method for Artificial Olfaction, and Lim et al, An optoelectronic nose for the detection of toxic gases. Nature Chemistry, 10.1038, 564-567, 2009, both of which are incorporated by reference herein in their entirety. Accordingly, when exposed a mixture of specific VOCs, an array of chemo-responsive dyes will change color in a distinct pattern that will be distinguishable from the color change using a different VOC mixture. Thus, with a large enough sensor array that includes a sufficient number of types of chemo-responsive dyes, a fingerprint of the VOCs contained in a particular patient's urine headspace gas can be detected.

### IV. Examples

The following examples disclose specific embodiments of the methods and compositions described herein and should be construed to encompass any and all variations that become evident as a result of the teaching provided herein.

### Example 1—Materials and Methods

Subjects. Inbred male C57BL/6J mice were bred in our laboratory or purchased from Jackson Laboratories. Mice were classified in one of two ways: as donor mice or as biosensor mice. The donor mice were treated as described below for urine and blood donation at 6-10 weeks old. Treated subjects had ad libitum access to food and water prior to and after treatment. Biosensor mice were bred in-house and began training at 5 weeks of age. Biosensors had ad libitum access to food but were restricted from water 23 hours a day during behavioral experiments that employed water as a reward. Biosensors received water during and after every behavioral experiment (having one hour to satiate). Because lack of adequate access to water will lead to significant loss of body condition, body mass of biosensor mice was monitored. All treatments, housing, care, and training was approved by the Monell Internal Animal Care and Use Committee protocols #1123 and #1174.

Treatments. For bioassay training and chemometric model building, 20 (each) mouse urine donors were injected (i.p.) with 250 μg/mL LPS in 0.01M phosphate-buffered saline (PBS) solution (2 mg/kg) or PBS solution only (0.2 mL each). For generalization trials and chemometric predictions, 10 (each) mouse urine donors were given 0.2 mL i.p. injections of either 6.25 μg/mL LPS (0.05 mg/kg), 250 μg/mL Poly(I:C) (2 mg/kg), or 250 μg/mL Pam3CSK4 (2 mg/kg) prepared in PBS solution.

Blood collection. Blood was collected at 2 h or 24 h post-treatment under 2% isoflurane anesthesia via tail vein in microcentrifuge tubes containing 3.8% w/v sodium citrate solution. Blood was immediately centrifuged, and plasma removed. Plasma was stored at −40° C. until use for enzyme-linked immunosorbent assays (ELISA).

Urine collection. Urine was collected daily from mice by application of gentle abdominal pressure, as described elsewhere (Millet et al. 2018; Yamazaki et al. 1983). Once collected, urine was immediately stored at −20° C. pending behavioral or chemometric analysis. Urine was collected daily from days 4 to 28 days following treatment. Urine samples were not collected prior to day 4 so that indications of fasting (food intake declines for about 48 hours following treatment with LPS for example) and anesthesia (isoflurane used for blood collection) were minimized in the odor profile of the urine samples.

ELISA. Assays for TNF and IL-1β were conducted using QUANTIKINE ELISA kits as directed by manufacturer (R&D Systems, Minneapolis, MN).

Behavioral Assay. Odor discrimination tasks by a panel of six trained biosensors were conducted using a Y-maze apparatus, in which mice are presented with a choice of two urine odors. The odor training, extinction, and generalization steps are described previously (Millet et al. 2018). Briefly, during training, biosensors were given a water reward for going to the arm of the Y-maze scented by urine odor from LPS (2 mg/kg)-treated donors (4-28 days post injection) when the other arm was scented by urine odors of PBS-treated donors (1-28 days post injection). Biosensors were not rewarded for identifying the urine odor from PBS-treated donors. When biosensors could reliably identify LPS urine odor, as evidenced by 80% correct selection of LPS-associated urine odor, extinction trials were initiated so that trained mice became accustomed to unrewarded training trials (regardless of correct choice). Critical generalization trials were then conducted with presentation of odors from novel donors (donors that did not provide urine for training trials). The first generalization trials were conducted with urines collected from novel LPS (2 mg/Kg) and PBS-treated donors. Having evidence from these validation trials that trained biosensors were selecting the maze arm containing LPS-derived odor in favor of PBS-derived odors on the basis of their training, further generalization trials were conducted using urine from donors receiving other treatments in order to test the two hypotheses.

Hypothesis 1. Variation in the volatile metabolome is controlled by the intensity of inflammation. Our lab and others have demonstrated that LPS impacts body odor in a way distinct from other immune stimuli (Arakawa et al. 2010; Gervasi et al. 2018; Kimball et al. 2014a; Millet et al. 2018). We hypothesized that large differences in the degree of immune activation could be coded differently in the body odor profile of the affected animal. To test whether alteration of volatiles differs by LPS dosage, we treated mice with two very different doses of LPS. Treatment urines derived from treatment with 0.05 mg/Kg LPS were presented in the Y-maze versus urine from a PBS-treated donor (not previously used during training). Generalization of the learned response to urine odors from donors receiving reduced LPS dosage (as evidenced by selection of LPS urines at a rate greater than 50%) would indicate that patterns of volatile odorants resulting from the two LPS dosages were perceptually similar.

Hypothesis 2. Variation in the volatile metabolome is controlled by pathogen-specific inflammation. Treatment urines derived from treatment with Pam3CSK4 or Poly(I:C) were presented separately in the Y-maze versus urine from a PBS-treated donor not previously used during training. Generalization of the learned response to urine odors from donors receiving different PAMPs (as evidenced by selection of LPS urines at a rate greater than 50%) would indicate that patterns of volatile odorants resulting from different their administration were perceptually similar to LPS-induced alterations of the volatile metabolome.

Behavioral data analysis. For each set of generalization trials, responses coded as correct/incorrect (where the incorrect answer was selection of PBS urine odor) from all biosensors were combined. Each data set, representing a unique generalization experiment, contained n≥75 total trials. Data were then subject to two-tailed binomial proportion hypothesis testing using the PROC FREQ function of SAS Studio. Significance was decided by exact test of binomial proportion.

Chemometric analyses. Urine samples from donors treated with low-dose LPS were not subjected to chemical analyses. Thus, only hypothesis 2 was tested using chemometric data. Model building and prediction followed precisely with behavioral assays. Just as biosensor mice were trained with urines from donors receiving either 2 mg/Kg LPS or PBS, data from analyses of these urines were used for linear discriminant analysis (LDA) modeling building. Similarly, as generalization trials were conducted with urines from donor's treatment with either Pam3CSK4 or Poly(I:C), data from analyses of these urines were entered into the LDA model for prediction.

Headspace GC/MS. Twenty-five μL samples of urine were placed in 20-mL headspace vials and fortified with 10 μL of an internal standard consisting of 1 ug/mL L-carvone in water (such that 100 ng was delivered to each sample). At least three urine samples (collected 4-28 days post-treatment) from each subject were analyzed by headspace gas chromatography/mass spectrometry (GC/MS). Quality control samples consisting of empty vials or vials containing only 100 ng of L-carvone were also analyzed throughout each chromatographic run consisting of 24 urine samples. Samples were analyzed using a HT3 dynamic headspace analyzer (Teledyne Tekmar) with a Supelco Trap K Vocarb 3000 thermal desorb trap (Sigma-Aldrich) attached to an ISQ GC/MS (Thermo Scientific) equipped with a single quadrupole mass spectrometer (Kimball et al. 2016a; Kimball et al. 2016b). Samples were incubated at 40° C. and swept with helium for 10 minutes at 75 mL/minute as volatile metabolites were collected on the thermal trap. After the sweep, the trap was heated to 260° C. and volatiles desorbed directly onto the gas chromatograph equipped with 30 m×0.25 μm Stabilwax-DA (Restek) capillary column. Split injections (5:1) were made with a column flow of 2.0 mL/minute and a split vent flow of 10.0 mL/minute. The GC oven began at 40° C. (3 min) and ramped to 260° C. at a rate of 7° C./minute. The mass spectrometer was operated in scan mode from 33-400 m/z. Tentative chromatographic peak identifications were based on the NIST Standard Reference Database.

Chemometric analyses. Chromatographic data were exported to MetAlign software for peak alignment and noise elimination (Lommen 2009). Resulting data were analyzed by MSClust for mass spectral extraction and assignment of a single peak response value based on the extracted selected ion monitoring (SIM) trace (Tikunov et al. 2012). Peak responses were normalized to the L-carvone response from each sample.

Data were separated into two groups. Group 1 (model set) contained data from mice treated with PBS or LPS. Group 2 (prediction set) contained data from mice treated with Pam3CSK4 or Poly(I:C). Subject means from multiple analyses were calculated for individual mice in Group 1. We performed stepwise linear discriminant analysis (LDA) model building for the model set using PROC STEPDISC in SAS to identify chromatographic peaks which contribute to the LPS vs PBS discrimination task. Using this model, contingency tables for PBS and LPS classifications were constructed using PROC DISCRIM with cross-validation to determine the overall validity and error rate of the model. Individual samples from Group 2 were then subjected to LDA classification using this model.

Results

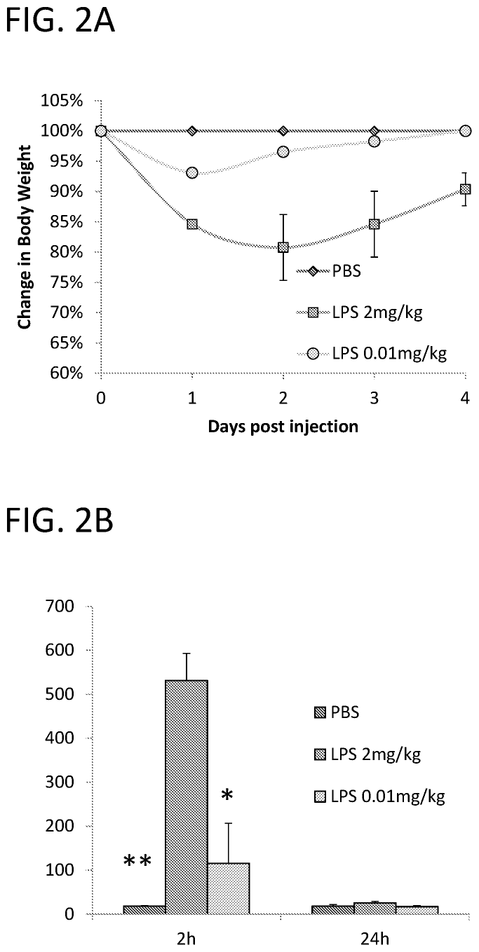

Treatment with PAMPs induce inflammation. Mice showed signs of inflammation for 24-72 hours following treatment with a PAMP. Effects included lethargy, reduced grooming, and anorexia. Mice treated with the reduced dose of LPS to test hypothesis 1 did not lose as much of their body mass as those treated with the higher dose (FIG. 2A). Furthermore, the inflammatory cytokines produced in response to 0.05 mg/kg LPS were lower than the 2 mg/kg counterparts (FIG. 2B).

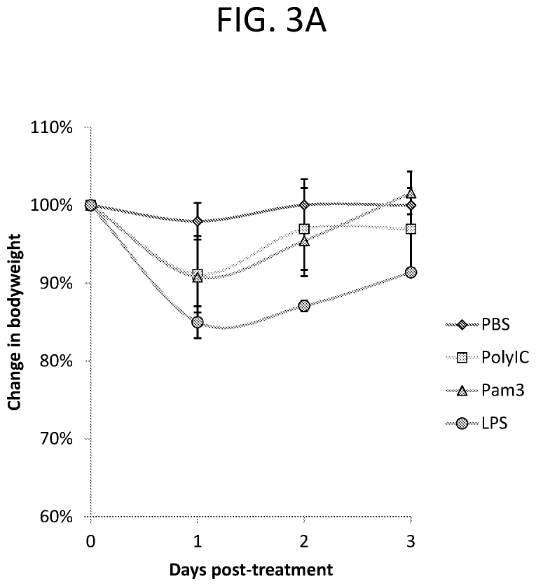

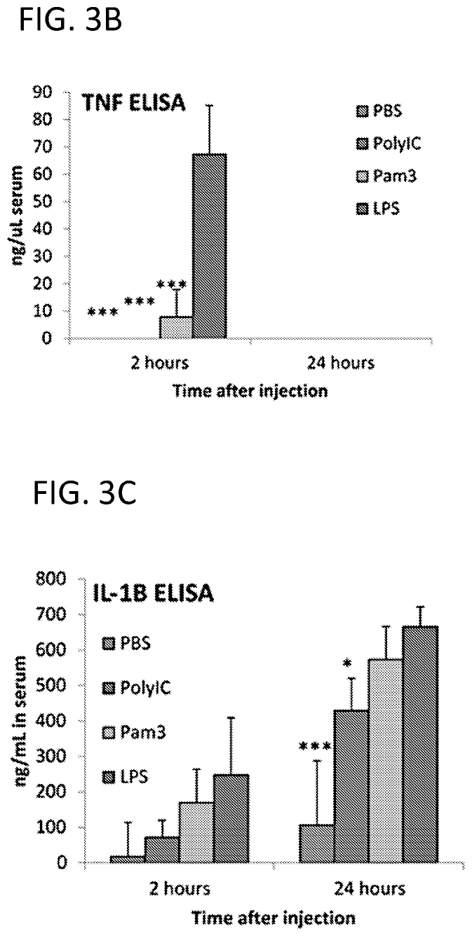

Mice treated with LPS, Pam3CSK4, and Poly(I:C) experienced reduced body mass 24 hours after treatment (FIG. 3A). This decrease of 10-15% body mass was most prominent in LPS-treated animals. LPS-treated animals were also slower to recover their pre-injection body weights. LPS-treated mice displayed a stronger TNF response than either PBS, Poly(I:C), or Pam3CSK4 treated mice (FIG. 3B). However, all three treatments produced an increase in IL-1β levels 24 hours after injection (FIG. 3C). It should be noted that while Poly(I:C) produced an increase in IL-1β compared to control, this increase was lower than the increase induced by LPS. Together, these data indicate that while equal treatments of all three PAMPs induced inflammation, the inflammatory response was strongest in the 2.0 mg/Kg LPS-treated mice.

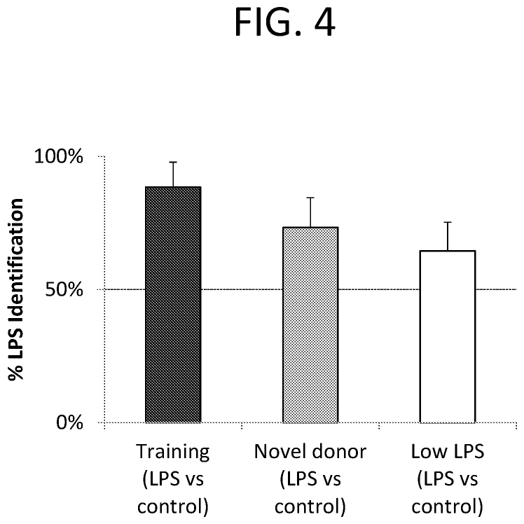

Hypothesis 1. Variation in the volatile metabolome is controlled by the intensity of inflammation. During training, biosensors identified the urine of 2 mg/Kg LPS-treated conspecifics (as opposed to control conspecifics) correctly 88% of the time (FIG. 4). This identification rate dropped to 73% during unrewarded generalization trials (i.e., validation) to novel individuals who received the same treatment. Validation trials with novel donors are essential to ensure the biosensors have genuinely learned the body odor induced by LPS administration, rather than merely learning individual donor identity during training. Biosensor mice trained with urine from donors treated with 2 mg/kg LPS reliably generalized this response to urine odor of mice treated with 0.05 mg/kg LPS (FIG. 4). As used herein the term “Generalize” means transfer of a learned response to novel stimuli. These data provide evidence against the hypothesis that the magnitude of inflammation dictates how inflammation information is coded in body odor.

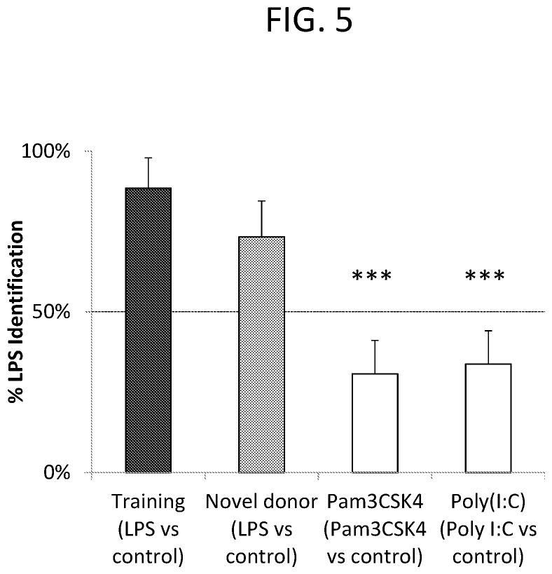

Hypothesis 2. Variation in the volatile metabolome is controlled by pathogen-specific inflammation. Biosensors did not generalize urine odors associated with treatments of Pam3CSK4 or Poly(I:C) to the learned odor of LPS treatment (FIG. 5). Biosensors generalized to Pam3CSK4-urine odor only 31% of the time, and to Poly(I:C) urine odor only 34% of the time. Both of these identification rates were significantly lower than that expected by chance (p=0.0011 and p=0.0049, respectively). This indicates that biosensors did not perceive the urine odor of either treatment to be similar to LPS urine odor.

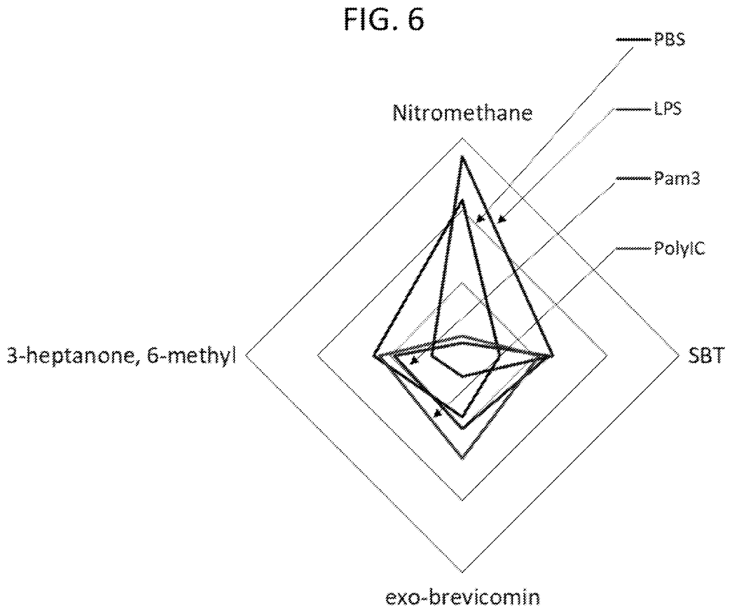

The LDA model constructed from 45 peaks identified in processed chromatograms yielded a four-peak model capable of discriminating between PBS and LPS samples. This model had an average squared canonical correlation of 0.87, which was not improved by further addition or removal of any predictors. Predictors were tentatively identified as nitromethane, exo-brevicomin, 2-sec-Butyl-4,5-dihydrothiazole (SBT), and 6-methyl-3-heptanone (FIG. 6). The cross-validation error rate for this model was 6.25%, meaning there was little error when sample means were held out of the model building one-at-a-time and samples reclassified (Table I).

TABLE I
Classification of samples from donors treated with Pam3CSK4
or Poly(I:C) using the linear discriminant analysis
(LDA) model built to discriminate lipopolysaccharide
(LPS) and phosphate buffered saline control (PBS).

| | No and % of Donors | | |
|---|---|---|---|
| Treatments | LPS | PBS | Probabilities¹ |
| Pam3CSK4 | 26 | 11 | P = 0.010 |
| | 70.27% | 29.73% | |
| Poly(I:C) | 17 | 15 | P = 0.430 |
| | 53.13% | 46.88% | |
| Total | 43 | 26 | |
| | 62.3% | 37.7% | |

¹P-values indicate significant difference from assumed distribution of 50% to each category expected by chance.

When this model was used to categorize individual Pam3CSK4-urine samples as either PBS or LPS, a significant portion were identified as LPS (26 of 37, p=0.010). However, when Poly(I:C) urine samples were classified as either PBS or LPS, there was a relatively even split between the two categories indicating that the pattern of Poly(I:C) volatiles differed from LPS-induced volatiles (Table I).

Previous studies demonstrate that various inflammatory stimuli differ in how they impact the mouse urinary odor profile (Kimball et al. 2014a; b; Millet et al. 2018). To explain this, we devised two hypotheses. One hypothesis was that differences in odor profile were due to the severity of inflammation. An alternative, but not mutually exclusive, hypothesis is that a qualitative difference in the inflammatory response is coded differently in the volatile metabolome as dictated by the cellular immune response.

We found no evidence for the first hypothesis as trained biosensors determined the resultant alterations of the volatile metabolome to be equivalent between the two LPS doses, despite the clearly demonstrated differences in intensity. Conversely, if alterations of the volatile metabolome are directed by the intracellular signaling pathway (hypothesis 2), biosensor mice would be expected to generalize their LPS training to urine odor from Poly(I:C)-treated donors (based on the shared TRIF pathway, FIG. 1). The LDA model would similarly be expected to classify Poly(I:C) donors to be in the LPS treatment group. However, both behavioral assay (FIG. 5) and LDA classification (Table I) concluded that the patterns of volatiles produced by LPS and Poly(I:C) were uniquely different. Hypothesis 2 would further predict that LPS and Pam3CSK4-treatment would produce similar alterations of the volatile metabolome. While this prediction was confirmed by results of the chemometric model (Table I), it was contradicted by the trained biosensors (FIG. 5). This may reflect the superior sensitivity of biosensor animals to trace volatile metabolites. Multidimensional gas chromatography combined with high resolution mass spectrometry may be necessary as this difference also indicates that the biosensors use volatiles other than those identified during model building for their discrimination tasks. Regardless, these results do not lend themselves to interpretation that alteration of the volatile metabolome is guided by stimulation of MyD88 and TRIF pathways.

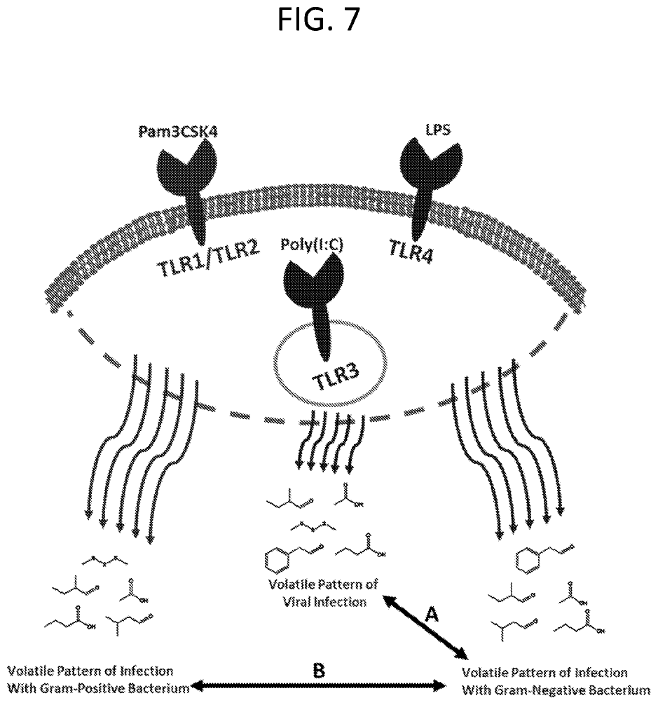

Results from behavioral assays indicate that activation of TLR2/1 or TLR3 produces a different urine odor profile relative to activation of TLR4 (FIG. 7). This suggests that simultaneous activation of both MyD88-mediated and TRIF-mediated inflammation has effects on body odor unlike that when either pathway is activated individually. This would further suggest that neither MyD88 nor TRIF are solely responsible for the LPS-mediated effect on the body odor volatiles. Although chemometric results did not confirm differences between TLR2/1 and TLR4 activation (contrary to behavioral results), both results indicate that bacterial and viral infection will result in uniquely different alterations of the volatile metabolome that allows for diagnosis of RNA viral infection from infections with gram positive or gram-negative bacteria.

Some of the predictors identified for the discrimination task between PBS-treated urine samples and LPS-treated urine samples have previously been noted for their importance in murine social communication (e.g., SBT) and identified as volatile molecules of interest in studies of inflammation-induced volatile metabolites (Gervasi et al. 2018; Kimball et al. 2016a; Millet et al. 2018). SBT has been previously found to be affected by diet, MHC type, and inflammatory mediators (Gervasi et al. 2018; Kwak et al. 2008; Millet et al. 2018). 6-Methyl-3-heptanone may be related to the mouse pheromone 6-hydroxyl-6-methyl-3-heptanone (HMH), which accelerates puberty in female mice (Novotny et al. 1999) and which previous studies have found decreases with LPS treatment (Gervasi et al. 2018).

Our results have significant implications for volatile metabolomic analyses in differential diagnostic tasks. As discussed above, more rapid differential diagnosis would be of great benefit to patients suffering from a variety of febrile diseases, such as SERS. Our results indicate that analysis of urine volatiles allows for rapid differential diagnosis between pathogens, or at minimum, between certain classifications of pathogens (FIG. 7). This allows for more targeted treatments in cases where differential diagnosis is currently difficult. Based on this present study, we can conclude there is at least more than one type of inflammatory odor profile.

Each and every patent, patent application, and publication, including websites cited throughout specification are incorporated herein by reference. In addition, U.S. Provisional Patent Application No. 63/139,515, filed Jan. 20, 2021 is incorporated by reference in its entirety. While the invention has been described with reference to particular embodiments, it will be appreciated that modifications can be made without departing from the spirit of the invention. Such modifications are intended to fall within the scope of the appended claims.

### REFERENCES

Alexopoulou L, et al. 2001. Recognition of double-stranded RNA and activation of NF-kappa B by Toll-like receptor 3. Nature 413: 732-738.
Aliprantis A O, et al. 1999. Cell activation and apoptosis by bacterial lipoproteins through toll-like receptor-2. Science 285: 736-739.
Amann A, et al. 2014. The human volatilome: volatile organic compounds (VOCs) in exhaled breath, skin emanations, urine, feces and saliva. J. Breath Res. 8.
Angus D C, van der Poll T. 2013. Severe sepsis and septic shock. N Engl J Med 369: 840-851.
Arakawa H, et al. 2011a. The role of neuroinflammation in the release of aversive odor cues from footshock-stressed rats: Implications for the neural mechanism of alarm pheromone. Psychoneuroendocrinology 36: 557-568.
Arakawa H, et al. 2009a. Acute Illness Induces the Release of Aversive Odor Cues From Adult, but Not Prepubertal, Male Rats and Suppresses Social Investigation by Conspecifics. Behavioral Neuroscience 123: 964-978.
Arakawa H, et al. 2010. Sickness-related odor communication signals as determinants of social behavior in rat: a role for inflammatory processes. Horm Behav 57: 330-341.
Arakawa H, et al. 2009b. Central infusion of interleukin-1 receptor antagonist blocks the reduction in social behavior produced by prior stressor exposure. Physiology & Behavior 98: 139-146.
Arakawa H, et al. 2011b. From models to mechanisms: Odorant communication as a key determinant of social behavior in rodents during illness-associated states. Neuroscience and Biobehavioral Reviews 35: 1916-1928.
Bajtarevic A, et al. 2009. Noninvasive detection of lung cancer by analysis of exhaled breath. BMC Cancer 9: 348.
Baldwin A S. 1996. The NF-kappa B and I kappa B proteins: New discoveries and insights. Annual Review of Immunology 14: 649-683.
Buie P S, et al. 2005. Influence of antibiotic therapy on mortality of critical surgical illness caused or complicated by infection. Surg Infect (Larchmt) 6: 41-54.
Beeson P B. 1947. Tolerance To Bacterial Pyrogens: I. Factors Influencing Its Development. J Exp Med 86: 29-38.
Bernabei M, et al. 2008. A preliminary study on the possibility to diagnose urinary tract cancers by an electronic nose. Sensors and Actuators B-Chemical 131: 1-4.
Bhaskaran D, et al. 2019. Diagnostic tools used in the evaluation of acute febrile illness in South India: a scoping review. BMC Infectious Diseases 19: 970.
Bijland L R, et al. 2013. Smelling the diagnosis: a review on the use of scent in diagnosing disease. Neth J Med 71: 300-307.
Brightbill H D, et al. 1999. Host defense mechanisms triggered by microbial lipoproteins through toll-like receptors. Science 285: 732-736.
Burdette S D, Bernstein J M. 2007. Does the nose know? The odiferous diagnosis of Clostridium difficile-associated diarrhea. Clin Infect Dis. United States. p. 1142.
Dellinger R P, et al. 2013. Surviving Sepsis Campaign: International Guidelines for Management of Severe Sepsis and Septic Shock: 2012. Critical Care Medicine 41: 580-637.
Dittrich S, et al. 2016. Target Product Profile for a Diagnostic Assay to Differentiate between Bacterial and Non-Bacterial Infections and Reduce Antimicrobial Overuse in Resource-Limited Settings: An Expert Consensus. PLOS ONE 11: e0161721.
Dospinescu, V-M et al, July 2020, Sniffing Out Urinary Tract Infection—Diagnosis Based on Volatile Organic Compounds and Smell Profile, Biosensors 2020, 10: 83; doi:10.3390/bios10080083
Geppert T D, et al. 1994. Lipopolysaccharide Signals Activation of Tumor-Necrosis-Factor Biosynthesis through the RAS/RAF-1/MEK/MAPK Pathway. Molecular Medicine 1: 93-103.
Gervasi S S, et al. 2018. Sharing an environment with sick conspecifics alters odors of healthy animals. Scientific Reports 8.
Greiter M B, et al. 2010. Differences in Exhaled Gas Profiles Between Patients with Type 2 Diabetes and Healthy Controls. Diabetes Technology & Therapeutics 12: 455-463.
Jezierski T, et al. 2015. Study of the art: canine olfaction used for cancer detection on the basis of breath odour. Perspectives and limitations. Journal of Breath Research 9.
Kapasi A J, et al. 2016. Host Biomarkers for Distinguishing Bacterial from Non-Bacterial Causes of Acute Febrile Illness: A Comprehensive Review. PLOS ONE 11: e0160278.
Kavaliers M, Colwell D D. 1995. Odors of Parasitized Males Induce Aversive Responses in Female Mice. Animal Behaviour 50: 1161-1169.
Kavaliers M, et al. 1998. Parasitized female mice display reduced aversive responses to the odours of infected males. Proceedings of the Royal Society B-Biological Sciences 265: 1111-1118.
Kimball B A. 2016. Volatile metabolome: problems and prospects. Bioanalysis 8: 1987-1991.
Kimball B A, et al. 2016a. Brain Injury Alters Volatile Metabolome. Chemical Senses 41: 407-414.
Kimball B A, et al. 2014a. Immunization alters body odor. Physiol Behav 128: 80-85.
Kimball B A, et al. 2014b. Immunization alters body odor. Physiology & Behavior 128: 80-85.
Kimball B A, et al. 2016b. Alterations of the volatile metabolome in mouse models of Alzheimer's disease. Scientific Reports 6.
Kumar A, et al. 2006. Duration of hypotension before initiation of effective antimicrobial therapy is the critical determinant of survival in human septic shock. Crit Care Med 34: 1589-1596.
Kwak J, et al. 2008. Genetically-Based Olfactory Signatures Persist Despite Dietary Variation. Plos One 3.
Lommen A. 2009. MetAlign: Interface-Driven, Versatile Metabolomics Tool for Hyphenated Full-Scan Mass Spectrometry Data Preprocessing. Analytical Chemistry 81: 3079-3086.
Lu Y C, et al. 2008. LPS/TLR4 signal transduction pathway. Cytokine 42: 145-151.
Mahoney A, et al. 2013. Giant African Pouched Rats As Detectors Of Tuberculosis In Human Sputum: Comparison Of Two Techniques For Sputum Presentation. Psychological Record 63: 21-26.
Mgode G F, et al. 2012. Mycobacterium tuberculosis volatiles for diagnosis of tuberculosis by Cricetomys rats. Tuberculosis 92: 535-542.
Millet P, et al. 2018. Cytokine contributions to alterations of the volatile metabolome induced by inflammation. Brain Behavior and Immunity 69: 312-320.
Morrell M, et al. 2005. Delaying the empiric treatment of candida bloodstream infection until positive blood culture results are obtained: a potential risk factor for hospital mortality. Antimicrob Agents Chemother 49: 3640-3645.
Murugathas, T et al, 2019 Biosensing with Insect Odorant Receptor Nanodiscs and Carbon Nanotube Field-Effect Transistors, ACS. Appl. Mater. Interfaces, 11(9):9530-9538
Novotny M V, et al. 1999. Positive identification of the puberty-accelerating pheromone of the house mouse: the volatile ligands associating with the major urinary protein. Proc. Royal Soc. B-Biological Sciences 266: 2017-2022.
Penn D, et al. 1998. Influenza infection neutralizes the attractiveness of male odour to female mice (Mus musculus). Ethology 104: 685-694.
Phillips M, et al. 2012. Point-of-care breath test for biomarkers of active pulmonary tuberculosis. Tuberculosis 92: 314-320.
Raetz C R H, Whitfield C. 2002. Lipopolysaccharide endotoxins. Annual Review of Biochemistry 71: 635-700.
Sun, M et al, November 2016, Bioelectronic Nose Using Odorant Binding Protein-Derived Peptide and Carbon Nanotube Field-Effect Transistor for the Assessment of Salmonella Contamination in Food, Anal. Chem. 88, 23, 11283-11287
Tikunov Y M, Laptenok S, Hall R D, Bovy A, de Vos R C H. 2012. MSClust: a tool for unsupervised mass spectra extraction of chromatography-mass spectrometry ion-wise aligned data. Metabolomics 8: 714-718.
van Oort P M, et al. 2018. The potential role of exhaled breath analysis in the diagnostic process of pneumonia—a systematic review. Journal of Breath Research 12.WHO. 2018. http://unitaid.org/assets/Fever_diagnostic_technology_and_market_landscape. pdf.
Yamazaki K, et al. 1983. Sensory Distinction Between H-2B and H-2BM1 Mutant Mice. Proc. Natl. Acad. Sci. USA—Biological Sciences 80: 5685-5688.

1. An assay for determining a source of infection in a mammalian subject comprising contacting a biological sample from said subject with a diagnostic reagent that detects at least one, or a pattern of multiple, volatile organic compounds (VOC), wherein detection of said compound or a pattern of said VOC indicates a single source of infection selected from a Gram-positive bacterial infection, a Gram-negative bacterial infection, or a viral infection.

2. The assay according to claim 1, wherein the VOC is 3-methyl-4-hexen-2-one.

3. The assay according to claim 1, wherein the VOC is acetophenone.

4. The assay according to claim 1, wherein the VOC is 2-ethyl-1-hexanol.

5. The assay according to claim 1, wherein the VOCs are 3-methyl-4-hexen-2-one and acetophenone.

6. The assay according to claim 1, wherein the VOCs are 3-methyl-4-hexen-2-one and 2-ethyl-1-hexanol.

7. The assay according to claim 1, wherein the VOCs are 2-ethyl-1-hexanol and acetophenone.

8. The assay according to claim 1, wherein the VOCs are 2-ethyl-1-hexanol, acetophenone, and 3-methyl-4-hexen-2-one.

9. The assay according to claim 1, wherein the detection of increased acetophenone relative to a standard for viral infection or Gram-positive infection indicates that the infection is a Gram-negative bacterial infection.

10. The assay according to claim 1, wherein the detection of increased 2-ethyl-1-hexanol relative to a standard for viral infection or Gram-negative infection indicates that the infection is a Gram-positive bacterial infection.

11. The assay according to claim 1, wherein the detection of increased 3-methyl-4-hexen-2-one relative to a standard for Gram-negative infection or Gram-positive infection relative to a standard indicates that the infection is a viral infection.

12. The assay according any one of claims 1 to 11, wherein the pattern of VOC levels is as demonstrated in FIG. 8.

13. The assay according to any one of claims 1 to 12, wherein the biological sample is urine, saliva, tears, nasal secretions, or blood.

14. The assay according to any one of claims 1 to 13, wherein the diagnostic reagent comprises a compound or substance that reacts with one or more selected VOC upon contact with the sample containing the VOCs to produce a characteristic signal.

15. The assay according to claim 14, wherein the signal is a color change or colorimetric signal, a fluorescent signal, an enzymatic signal or a chromogen.

16. The assay according to claim 14 or claim 15, wherein the reagent comprises a dipstick impregnated with the VOC-reactive compound or substance.

17. The assay according to claim 16, wherein the VOC-reactive compound or substance is a chemical reactant or an enzyme, or a chemical reactant or enzyme bound to a signally molecule.

18. The assay according to claim 1, wherein the VOC-reactive compound or substance is 2,4-dinitrophenylhydrazine.

19. A diagnostic device configured for detection of a Gram-positive bacterium, a Gram-negative bacterium or a virus in a test sample.

20. The device according to claim 19, wherein said device is configured for detection of a selected VOC or pattern of VOC in a test sample via a competition assay, a sandwich assay, a displacement assay, electrochemical detection, colorimetric detection, enzymatic detection, an enzyme-linked immunosorbent assay (ELISA), or a dipstick assay.

21. The device according to claim 19, wherein said device comprises a compound or substance that reacts with one or more selected VOC upon contact with the sample containing the VOCs to produce a characteristic signal.

22. The device according to claim 21, wherein the VOC-reactive compound or substance is conjugated, attached or immobilized onto a solid substrate.

23. The device according to claim 22, wherein the solid substrate is a dipstick or a nanoparticle.

24. The device according to claim 21, wherein at least a portion of the VOC reactive compound or substance is conjugated or associated with a detectable label.

25. The device according to claim 19, comprising: a sample pad; the VOC reactive compound or substance immobilized on a first surface; and wherein the sample pad is in operable fluid communication with the first surface.

26. The device according to claim 19, wherein the first surface is a porous surface.

27. The device according to claim 19, wherein the first surface comprises nitrocellulosic material, polyvinylidene fluoride (PVDF), polyethylene material, nylon, cellulose acetate, polyester material, polyethersulfone (PES), or polysulfone.
